(19) United States
(12) Patent Application Publication
(10) Pub. No.: US 2021/0299039 A1
(43) Pub. Date: Sep. 30, 2021

(54) COMPOSITIONS AND METHODS FOR TREATING OCULAR DISEASE BY CONTACT LENS MEDIATED DRUG DELIVERY

(71) Applicant: EyeGate Pharmaceuticals, Inc., Waltham, MA (US)

(72) Inventors: Brenda K. Mann, Salt Lake City, UT (US); Darren Stirland, Salt Lake City, UT (US); Michael Manzo, Beverly, MA (US); Heather Sheardown, Ontario (CA); Talena Rambarran, Ontario (CA); Lina Liu, Ontario (CA)

(73) Assignee: EyeGate Pharmaceuticals, Inc., Waltham, MA (US)

(21) Appl. No.: 17/209,645

(22) Filed: Mar. 23, 2021

Related U.S. Application Data
(60) Provisional application No. 62/994,456, filed on Mar. 25, 2020.

(51) Int. Cl. A61K 9/00; A61K 31/573; A61K 47/34; A61K 47/32

(52) U.S. Cl. CPC A61K 9/0051; A61K 47/32; A61K 47/34; A61K 31/573

(57) ABSTRACT

The disclosure provides an ocular drug delivery system that includes a contact lens and a drug delivery portion, which may include a compound having the formula X—(CH2)n-Z, wherein X is a photocrosslinkable group. Advantageously, the drug delivery portion may aid in loading a high concentration of a negatively-charged therapeutic agent into the ocular drug delivery system. Additionally, the disclosed ocular drug delivery system may aid in controlled delivery of the negatively-charged therapeutic agent to the eye of a patient over a period of about 4 to about 24 hours.

### CROSS REFERENCE TO RELATED APPLICATIONS

This application claims the benefit of U.S. Provisional Application No. 62/994,456, filed Mar. 25, 2020, entitled “COMPOSITIONS AND METHODS FOR TREATING OCULAR DISEASE BY CONTACT LENS MEDIATED DRUG DELIVERY,” the entire contents of which are incorporated herein by reference.

### FIELD OF THE DISCLOSURE

The disclosure relates to compositions and methods for treating ocular diseases by contact lens mediated delivery of therapeutic agents. In particular, the disclosure relates to a contact lens having a drug delivery portion that includes a molecule that provides high loading capacity and controlled delivery of negatively-charged therapeutics to the eye.

### BACKGROUND OF THE DISCLOSURE

Contact lenses designed to deliver drugs either release all of the drug very quickly (within an hour or two) or deliver the drug over many days. In the first case, the concentration of drug in the contact lens must be limited to avoid toxicity that can occur when such a high drug dose is delivered so quickly. Additionally, due to high clearance of the drug from the surface of the eye, much of what is delivered is quickly eliminated. In the second case, the user must wear the contact lens continuously for many days, which can have detrimental effects to the eye due to oxygen permeability issues (e.g., reduced oxygen transport to the cornea). Therefore, there is a need for a contact lens that can be loaded with a high concentration of drug that is delivered over the course of about 4 to about 24 hours.

### SUMMARY OF THE DISCLOSURE

The disclosure provides compositions and methods for treating ocular diseases by contact lens mediated delivery of therapeutic agents. In particular, the disclosure relates to a contact lens having a drug delivery portion including a molecule that provides high loading capacity and controlled delivery of therapeutic agents (e.g., negatively-charged therapeutic agents) to the eye over a short period of time (e.g., less than a day).

In one aspect, the disclosure provides a contact lens that includes a drug delivery portion having a covalently crosslinked polymer; a monomer having the formula X—(CH₂)ₙ—Z, wherein X is a crosslinkable group; n is 2, 3, or 4; Z is a morpholino, imidazole, or piperazine group; wherein the monomer is covalently linked to the crosslinked polymer through X; and a therapeutic molecule having a negative charge; where the contact lens has from about 20 wt % to about 40 wt % water.

In some embodiments, X is a methacryl, an acryl, a methacrylamide, an acrylamide, or a vinyl group.

In some embodiments, the monomer is at a concentration of about 5 wt % to about 20 wt %.

In some embodiments, the polymer is covalently crosslinked using light. In some embodiments, the light has a wavelength of about 200 nm to about 400 nm.

In some embodiments, the therapeutic molecule is a corticosteroid.

In some embodiments, the therapeutic molecule is a dexamethasone derivative.

In some embodiments, the therapeutic molecule has a phosphate group.

In some embodiments, the therapeutic molecule has a concentration of about 1% to about 5%.

In some embodiments, the contact lens has a thickness between about 0.1 mm and about 0.5 mm.

In some embodiments, the polymer comprises HEMA.

In some embodiments, the polymer comprises a silicone macromer.

In some embodiments, the therapeutic molecule is incorporated into the drug delivery portion prior to crosslinking.

In some embodiments, the therapeutic molecule is incorporated into the drug delivery portion by soaking the contact lens in a solution containing the therapeutic molecule.

In some embodiments, the therapeutic molecule in the solution is at a concentration of about 5 to about 80 mg/ml.

In some embodiments, the therapeutic molecule in the solution is at a concentration of about 5 to about 40 mg/ml.

In some embodiments, the therapeutic molecule is dexamethasone phosphate.

In some embodiments, the therapeutic molecule in the solution is at a concentration of about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 mg/ml.

In some embodiments, the therapeutic molecule is dexamethasone phosphate.

In some embodiments, the therapeutic molecule is incorporated into the drug delivery portion prior to crosslinking and also by soaking the contact lens in a solution containing the therapeutic molecule, optionally, the therapeutic molecule is dexamethasone phosphate.

In some embodiments, the drug delivery portion is a circumferential portion of the contact lens.

### Definitions

Unless defined otherwise, all technical and scientific terms used herein have the meaning commonly understood by a person skilled in the art to which this disclosure belongs. The following references provide one of skill with a general definition of many of the terms used in this disclosure: The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term about.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “agent” or “therapeutic agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease or a symptom thereof.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “co-administering,” or “co-administration,” and the like refers to the act of administering two or more agents (e.g., an antibiotic or therapeutic agent with a synergistic agent), compounds, therapies, or the like, at or about the same time. The order or sequence of administering the different agents of the disclosure, e.g., antibiotics or synergistic agent may vary and is not confined to any particular sequence. Co-administering may also refer to the situation where two or more agents (e.g., an antibiotic or therapeutic agent with a synergistic agent) are administered via different parts of a contact lens as described herein, e.g., where a first agent is administered by a drug delivery portion in a central portion of a contact lens and a second agent is administered by a drug delivery portion in a peripheral portion of a contact lens.

By “subject” is meant a mammal, including, but not limited to, a human or non-human mammal, such as a bovine, canine equine, feline, ovine, or primate.

A “therapeutically effective amount” is an amount sufficient to effect beneficial or desired results, including clinical results. An effective amount can be administered in one or more administrations (e.g., on one or more contact lenses).

As used herein, the terms “treat,” treating,” “treatment,” and the like refer to reducing or ameliorating an ocular disorder and/or symptoms (e.g., inflammation, bacterial infection, blepharitis, and the like) associated therewith. It will be appreciated that, although not precluded, treating a disorder or condition does not require that the disorder, condition or symptoms associated therewith be completely eliminated.

The term “pharmaceutically acceptable” as used herein, refers to a material, (e.g., a carrier or diluent), which does not abrogate the biological activity or properties of the compounds described herein, and is relatively nontoxic (i.e., the material is administered to an individual without causing undesirable biological effects or interacting in a deleterious manner with any of the components of the composition in which it is contained).

The phrase “pharmaceutically acceptable carrier, excipient, or diluent” is art recognized and includes a pharmaceutically acceptable material, composition or vehicle, suitable for administering compounds of the present disclosure to mammals. As used herein, the term “pharmaceutically acceptable” means being approved by a regulatory agency of the Federal or a state government or listed in the U.S. Pharmacopia, European Pharmacopia or other generally recognized pharmacopeia for use in mammals, e.g., humans.

Where applicable or not specifically disclaimed, any one of the embodiments described herein are contemplated to be able to combine with any other one or more embodiments, even though the embodiments are described under different aspects of the disclosure. These and other embodiments are disclosed and/or encompassed by, the following Detailed Description.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1 is the chemical structure of the silicone macromer used in some of the formulations.

FIG. 2 shows a comparison of the modulus and maximum strength from tensile testing of a silicone-based hydrogel formulation that contains the MPMA monomer (formulation #2) and without the MPMA monomer (formulation #5).

FIG. 3 shows the water content at equilibrium of several hydrogel formulations, silicone-based and HEMA-based, with or without the MPMA monomer.

FIG. 4 shows the amount of dexamethasone phosphate, incorporated into hydrogels during crosslinking that is washed out of the hydrogels during extraction washes to remove unreacted polymer and monomer components.

FIG. 5 shows the amount of drug loaded into hydrogels, with and without MPMA monomer incorporated, by soaking the hydrogels in a drug solution for 48 hours.

FIGS. 6A and 6B show release of dexP from silicone-based hydrogels, with and without MPMA monomer incorporated, following an initial soak in a drug solution (A) or re-soaking in a drug solution after the first release to reload drug (B).

FIG. 7 shows the total amount of drug released and extracted from HEMA-based hydrogels, with and without MPMA monomer incorporated.

FIG. 8 shows the release of dexamethasone phosphate over time from HEMA-based hydrogels, with and without MPMA monomer incorporated.

FIG. 9 shows the release of prednisolone phosphate over time from HEMA-based hydrogels, with and without MPMA monomer incorporated.

FIG. 10 shows the release of metronidazole over time from HEMA-based hydrogels, with and without MPMA monomer incorporated.

FIG. 11 shows the release of tobramycin over time from HEMA-based hydrogels, with and without MPMA monomer incorporated.

FIG. 12 shows the experimental setup used to determine release of dexamethasone phosphate from HEMA-based hydrogels into and across sclera tissue into a PBS reservoir. A: expanded view to show the different components; B: fully assembled view.

FIG. 13 shows the amount of dexamethasone phosphate at different time points in the sclera tissue and the PBS reservoir below the tissue following release of the drug from HEMA-based hydrogel s.

FIG. 14 shows the amount of dexamethasone phosphate (dexP) loaded into contact lenses versus the concentration of drug in the loading solution.

FIG. 15 shows the amount of dexP released from contact lenses over 8 hours versus the concentration of drug in the loading solution.

FIG. 16 shows the amount of dexP loaded into contact lenses with MPMA incorporated using either dexP in the free acid form (EG dexP) or the disodium form (disodium dexP).

FIG. 17 shows the release of dexP from contact lenses with MPMA incorporated after loading the lenses using either dexP in the free acid form (blue circles) or the disodium form (orange circles).

FIGS. 18A and 18B show total concentration of dexamethasone (dex)+dexamethasone phosphate (dexP) in tissues after dexP-loaded contact lenses remained on the eyes of rabbits for 2, 4, or 8 hours. FIG. 18A is scaled to show the full amount in all tissues; FIG. 18B cuts off the top of the scale to better show the tissues with lower concentrations.

### DETAILED DESCRIPTION OF THE DISCLOSURE

The present disclosure is based, at least in part, on the discovery that a compound having the formula X—(CH₂)ₙ—Z, wherein X is a group capable of participating in photocrosslinking (including, but not limited to, (meth)acrylate, (meth)acrylamide, or vinyl); n is 2, 3, or 4; Z is a morpholino, imidazole, or piperazine group; and wherein the monomer is covalently linked to the matrix of a soft/hydrogel contact lens through X, may be included in an ocular drug delivery system (e.g., a drug delivery portion of a contact lens) to aid in loading a high concentration of a negatively-charged therapeutic and subsequently aid in controlled delivery of that therapeutic to the eye over a period of about 4 to about 24 hours, but especially over a period of about 8 to about 16 hours. The contact lenses (CLs) herein provide a number of advantages over the prior art, including: increased loading capacity of therapeutic molecule and controlled release of therapeutic molecule over time period for daily-wear CLs. Additionally, controlled release of a therapeutic molecule over about 4 to about 24 hours may facilitate targeting the molecule to posterior segments of the eye, including the vitreous humor, retina, choroid, and optic nerve.

The first polymer contact lenses became commonly available in the early 1960s and were made from a polymer called poly(methylmethacrylate) (PMMA). Lenses made of PMMA are called hard lenses. In 1979, the first rigid gas-permeable lenses (also known as RGPs) became available. These lenses are made from a combination of PMMA, silicones and fluoropolymers. This combination allows oxygen to pass directly through the lens to the eye, which makes the lens safer and more comfortable for the wearer.

The silicone in the silicone hydrogel lens has an impact on its rigidity and flexibility. The hydrogel component facilitates wettability and fluid transport, which aids in lens movement. Silicone hydrogel lenses generally have a higher modulus, and are therefore more rigid than standard hydroxyethylmethacrylate lenses. Traditional soft contacts are made from hydrogel polymers—soft, water-containing plastics. Hydroxyethyl methacrylate (HEMA) is used to make the hydrophilic polymer. These lenses rely on the amount of water in the polymer to regulate how much oxygen can pass through the lens.

HEMA-based polymers include, but are not limited to, Tefilcon, Tetrafilcon, Crofilcon, Helfilcon A/B, Mafilcon, Polymacon, Hioxifilcon B, Surfilcon A, Lidofilcon A, Lidofilcon B, Netrafilcon A, Hefilcon B, Alphafilcon A, Omafilcon A, Omafilcon B, Vasurfilcon A, Hioxifilcon A, Hioxifilcon D, Nelfilcon A, Hilafilcon A Hilafilcon B, Acofilcon A, Nesofilcon A, Bufilcon A, Deltafilcon A Phemfilcon, Bufilcon A, Perfilcon A, Etafilcon A, Focofilcon A, Ocufilcon B, Ocufilcon C, Ocufilcon D, Ocufilcon E Ocufilcon F, Phemfilcon A, Methafilcon A, Methafilcon B, and Vilfilcon A.

Silicone hydrogel polymers include, but are not limited to, Lotrafilcon A, Lotrafilcon B, Galyfilcon A, Senofilcon A, Senofilcon C, Sifilcon A, Comfilcon A, Enfilcon A, Balafilcon A, Delefilcon A, Narafilcon B, Narafilcon A, Stenfilcon A, Somofilcon A, Fanfilcon A, Samfilcon A, and Elastofilcon.

Therapeutic agents or therapeutic molecules may be those used to treat anterior and/or posterior ocular diseases or conditions. Therapeutic agents or therapeutic molecules that may be suitable for delivery via the contact lens of the instant disclosure include, but are not limited to, anionic antibiotics, anionic anti-inflammatories, and anionic anti-allergy medications. Anionic antibiotics include, but are not limited to, cefuroxime, penicillin g, oxacillin, cefoxitin, carbenicillin, ticarcillin disodium, fluoroquinolones including pefloxacin, delafloxacin, and levofloxacin, and peptides such as dermicidin and anionic defensins.

Anionic anti-inflammatories include, but are not limited to, corticosteroid ester salts such as prednisolone phosphate, prednisolone sulfate, methylprednisolone phosphate, methylprednisolone sulfate, hydrocortisone phosphate, hydrocortisone sulfate, betamethasone phosphate, betamethasone sulfate, dexamethasone phosphate, dexamethasone sulfate, triamcinolone acetonide phosphate, and desonide phosphate.

A pro-drug or salt of a drug that renders the drug anionic could also be delivered. Additional therapeutics may be delivered by encapsulating the therapeutic compound in an anionic surfactant. Anionic surfactants contain anionic functional groups at their head, such as sulfate, sulfonate, phosphate, and carboxylates. Prominent alkyl sulfates include ammonium lauryl sulfate, sodium lauryl sulfate (sodium dodecyl sulfate, SLS, or SDS), and the related alkyl-ether sulfates sodium laureth sulfate (sodium lauryl ether sulfate or SLES), and sodium myreth sulfate. Other anionic surfactants include, but are not limited to, docusate (dioctyl sodium sulfosuccinate), perfluorooctanesulfonate (PFOS), perfluorobutanesulfonate, alkyl-aryl ether phosphates, alkyl ether phosphates, carboxylates such as sodium stearate, sodium lauroyl sarcosinate and carboxylate-based fluorosurfactants such as perfluorononanoate, perfluorooctanoate (PFOA or PFO).

Other therapeutic agents that may be suitable for delivery include peptides, oligos, proteins, siRNA, anti-angiogenic factors, and anti-apoptosis factors.

It is contemplated within the scope of the disclosure that a therapeutic agent or a therapeutic molecule may be present at a concentration of about 5 to about 80 mg/ml, or about 5 to about 40 mg/ml, or about 5 to about 30 mg/ml, or about 5 to about 20 mg/ml, or about 5 to about 10 mg/ml.

It is contemplated within the scope of the disclosure that a therapeutic agent or a therapeutic molecule may be present at a concentration of about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 mg/ml.

In some embodiments, the therapeutic agent or therapeutic molecule is dexamethasone phosphate.

According to the techniques herein, the disclosure provides a contact lens having a drug delivery portion, which may include all of the contact lens, or a portion thereof. In embodiments, the drug delivery portion may be a central portion of the contact lens. In embodiments, the drug delivery portion may be a circumferential portion of the contact lens (e.g., an annulus). It is contemplated within the scope of the disclosure that the drug delivery portion may be associated with the above-described monomer, which may facilitate increased delivery of a therapeutic agent associated with the drug delivery portion, leading to decreased potential for toxicity to the cornea or lens.

### EXAMPLES

The present disclosure is further illustrated by the following examples, which should not be construed as limiting. The contents of all references, published patents and patent applications cited throughout the application are hereby incorporated by reference. Those skilled in the art will recognize that the disclosure may be practiced with variations on the disclosed structures, materials, compositions and methods, and such variations are regarded as within the scope of the disclosure.

### Example 1: Hydrogel Formation

Various formulations of soft contact lens materials (hydrogels) were made with 3-(N-morpholino)propyl methacrylate (MPMA) as the monomer to aid with loading and release of drug. The formulations were either silicone-based or HEMA-based, and varied the concentration of MPMA (0-20%), photoinitiator (0.4-3%), and dexamethasone phosphate (0-5%). The final concentration of components of some exemplary formulations are shown in Table 1. For each formulation, components were mixed in the presence of an alcohol (methanol, ethanol, and/or hexanol) and transferred to a prepared UV-transparent polyester mold (52 mm×57 mm, thickness of 0.25 or 0.5 mm). The mold was then exposed to UV light (365 nm) for 5 minutes on each side to crosslink the material and form a hydrogel.

TABLE 1
Components of various formulations of soft contact lens hydrogel materials made with MPMA
monomer. Amounts of components are listed as w/w % in the final crosslinked material.

| Component | # 1 | # 2 | # 3 | # 4 | # 5 | # 6 |
|---|---|---|---|---|---|---|
| Lotrafilcon A | 81.6 | — | — | 78.2 | — | — |
| EGDMA | 1.6 | 5.0 | 3.4 | 1.6 | 1.0 | 3.4 |
| HEMA | — | — | 80.8 | — | — | 80.8 |
| DMA | — | 17.0 | — | — | 21.0 | — |
| Tris | — | 36.0 | — | — | 44.0 | — |
| PEGm | — | 6.0 | — | — | 8.0 | — |
| ACR | — | 10.0 | — | — | 13.0 | — |
| xlinker | — | 10.0 | — | — | 12.0 | — |
| 1-HCHPK | 0.4 | 1.0 | 0.8 | 0.4 | 1.0 | 1.0 |
| MPMA | 16.3 | 14.0 | 15.0 | 15.6 | — | 15.9 |
| dexP | — | — | — | 4.2 | — | — |

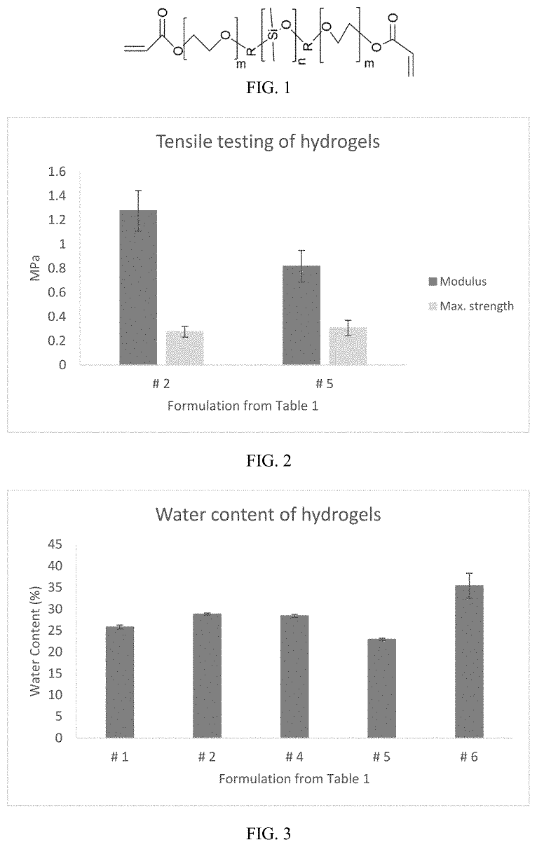

EGDMA: ethylene glycol dimethacrylate; HEMA: 2-hydroxyethyl methacrylate; DMA: dimethylacetamide; Tris: 3-[Tris(trimethylsiloxy)silyl]propyl methacrylate; PEGm: poly(ethylene glycol) methyl ether methacrylate; ACR: α-acrylate-ω-propylheptamethyltrisiloxy-(oligoethyleneglycol) or Silmer ACR A008 UP from Siltech Corp, MW 813.8 g/mol (surfactant); xlinker: silicone macromer, MW 1717 g/mol, see FIG. 1; 1-HCHPK: 1-hydroxycyclohexyl
phenyl ketone (photoinitiator); MPMA: 3-(N-morpholino)propyl methacrylate; dexP: dexamethasone phosphate. “—” indicates that component was not used in the formulation.

Each of the formulations in the table above resulted in a crosslinked hydrogel that could be removed from the mold.

Crosslinked hydrogels of Formulation 1 or 3 were also made with 3-(N-morpholino)propyl acrylamide, 3-(N-morpholino)propyl acrylate, 2-(N-morpholino)ethyl acrylate, 2-(N-morpholino)ethyl methacrylate, 4-(N-morpholino)butyl methacrylate instead of MPMA, varying the concentration from 15-30 wt %.

### Example 2: Physical Properties of Hydrogels

Soft contact lens hydrogels were made using formulations 2 and 5 in Table 1, which have the same components except that #2 incorporates MPMA and #5 does not, to determine any effects of the monomer on the elastic modulus and maximum strength of the hydrogels. For this tensile testing, hydrogels were cut into dumbbell-shaped pieces (gauge area 3 mm×12.5 mm), placed in the grips of an Instron 4401 with 50N load cell, and extended (10 mm/min) until breaking. Formulations 1, 2, 4, 5, and 6 from Table 1 were additionally used to determine water content of the hydrogels. To determine equilibrium water content, 8 mm diameter discs of the hydrogels were cut and placed in DI water for at least 12 hours, blotted with a Kimwipe to remove excess fluid, and weighed. The gel pieces were then dried in a vacuum oven for at least 24 hours and re-weighed. The equilibrium water content was then determined as ([wet weight]−[dry weight])/[wet weight]×100%.

As seen in FIG. 2, incorporation of 14% MPMA increases the elastic modulus and decreases the maximum strength compared to materials without MPMA. However, these values are similar to other soft contact lens materials; thus, the presence of the monomer does not detrimentally affect the tensile properties of the hydrogel. As seen in FIG. 3, the equilibrium water content of the hydrogels varies from about 25-35%, depending on the formulation and whether it is silicone-based or HEMA-based. Although the presence of MPMA does lead to a slightly higher equilibrium water content of the hydrogels (formulation #2 vs #5 in FIG. 3), the water content is very similar for hydrogels with and without the MPMA, and these are all within the acceptable range for soft contact lenses.

### Example 3: Crosslinking Drug into Hydrogels

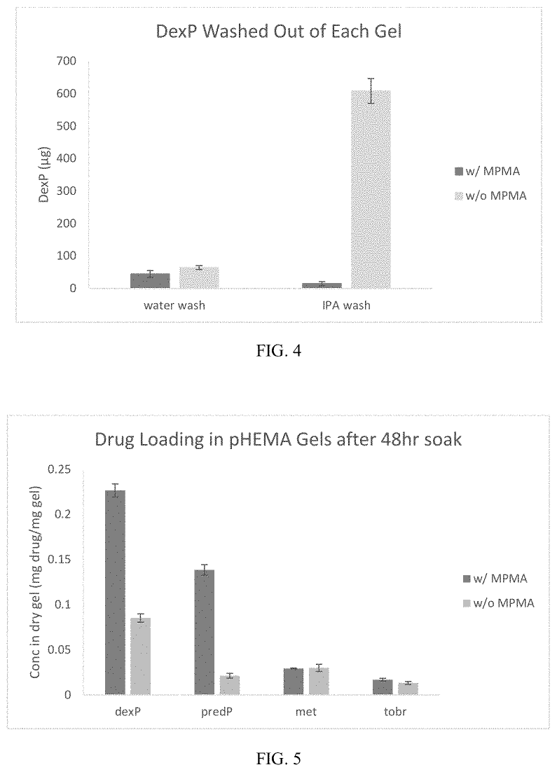

Soft contact lens hydrogels were made using formulation 4 in Table 1, wherein dexP was incorporated into the formulation prior to crosslinking to form the hydrogel. Hydrogels were made with and without the MPMA monomer to determine whether the MPMA would have a beneficial effect on keeping the dexP in the hydrogel during extraction washes of water and alcohol, as these extraction washes are typically done for soft contact lens hydrogel materials post-crosslinking to remove any unreacted/uncrosslinked components. Hydrogels were placed in deionized (DI) water for 35 minutes at 37° C./200 rpm, followed by 3 times in isopropyl alcohol for 25 minutes each at 37° C./200 rpm, then 2 additional times in DI water for 25 minutes each. The DI water washes and alcohol washes were collected and the amount of dexP was determined using HPLC-UV. As seen in FIG. 4, very little of the incorporated dexP was removed from the hydrogels during both the water and alcohol washes when MPMA was incorporated as compared to when MPMA was not incorporated into the hydrogels. These results demonstrate the beneficial effect of the monomer in allowing drug to be incorporated into the hydrogel pre-crosslinking and not being lost during extraction washes performed during manufacture of the contact lenses.

### Example 4: Loading by Soaking in Drug Solution

The HEMA-based hydrogels of formulation 5 in Table 1, were made with and without 15% MPMA. Unreacted components were extracted by washing in DI water for at least 3 hours, followed by washing in a 50:50 mixture of reagent alcohol and DI water for at least 3 hours, then washing again in DI water for at least 3 hours. Discs 15 mm in diameter were punched out and dried in a vacuum dessicator for at least 72 hours. The dry discs were weighed to get an initial weight without drug. Discs were then placed in a drug solution for 48 hours on a shaker at 200 rpm. Drug solutions used in this study can be found in Table 2. After 48 hours, the discs were removed from the drug solution, patted with a Kimwipe to remove solution from the surface, and then dried in a vacuum dessicator for at least 48 hours. The dry drug-loaded discs were weighed, and the amount of drug loaded was determined as the drug-loaded disc weight minus the initial weight.

TABLE 2
Drug solutions used to load pHEMA hydrogels. All
drug solutions were adjusted to pH 5.7 prior to use.

| | Concentration | | |
|---|---|---|---|
| | in solution | MW | Physiological |
| Drug | (mg/ml) | (g/mol) | charge |
| Dexamethasone | 40 | 472.4 | −2 |
| phosphate | | | |
| (dexP) | | | |
| Prednisolone | 40 | 486.4 | −2 |
| phosphate | | | |
| (predP) | | | |
| Metronidazole | 10 | 171.2 | 0 |
| Tobramycin | 40 | 467.5 | +5 |

As seen in FIG. 5, after a simple 48-hour soak in a drug solution, various drugs were loaded into the discs. For the two negatively charged drugs, dexP and predP, incorporation of MPMA in the crosslinked material significantly increased the amount of drug loaded compared to crosslinked material without the MPMA. The presence of MPMA in the material did not affect loading of the neutral-charge drug, metronidazole, nor the positively charged drug, tobramycin, for this initial 48-hour soak.

### Example 5: Drug Release from Silicone-Based Material

Silicone-based hydrogels similar to formulation 2 in Table 1, but with only 1% EGDMA, were made with and without 15% MPMA. Unreacted components were extracted as described in Example 4. Discs 10 mm in diameter were punched out and placed in a dexP solution for 5 days. The dexP-loaded discs were removed from the drug-loading solution, blotted to remove excess fluid, and placed in PBS to begin releasing the drug. At PBS was sampled at various timepoints and analyzed for dexP concentration using UV absorbance. After 12 hours of releasing drug, the discs were placed back in drug-loading solution overnight to re-load dexP, and the release of dexP from the re-loaded discs was performed in PBS as described.

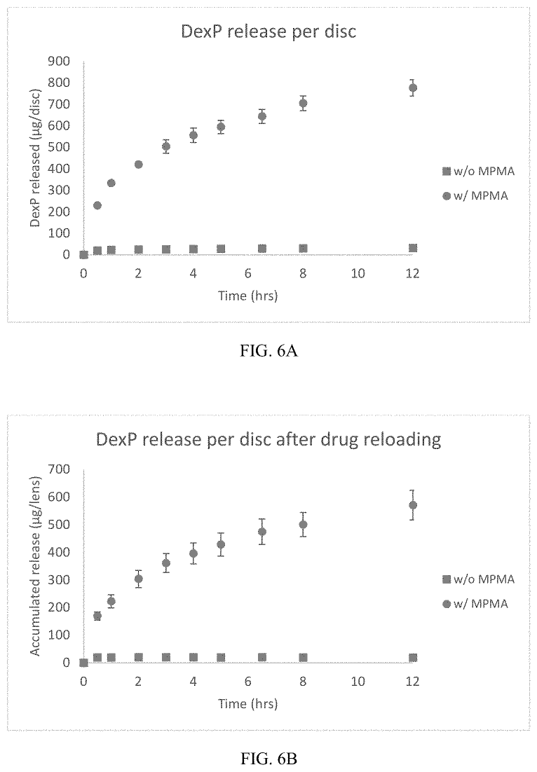

As seen in FIG. 6A, the total amount of dexP released was significantly greater when MPMA was incorporated in the crosslinked material. After re-loading the discs with dexP by soaking in the loading solution again, the materials with MPMA incorporated into them again exhibited a greater amount of dexP release, as shown in FIG. 6B.

### Example 6: Drug Release and Extraction from HEMA-Based Material

The drug-loaded discs described in Example 4 were placed back into their respective drug solutions for 24 hours on a shaker at 200 rpm at 37° C. to re-swell the discs. After 24 hours, discs were removed from the drug solution, blotted to remove excess solution, and placed in phosphate-buffered saline (PBS) to begin the release portion of the study. At 0.5, 1, 2, 4, 6, and 8 hours the PBS was removed and fresh PBS was added. After 24 hours, the PBS was removed and the discs were blotted, then placed in an extraction medium (80% reagent alcohol, 20% PBS) for 48 hours to remove any remaining drug. The removed PBS samples and extract media were analyzed for drug concentration using UV absorbance for dexP, predP, and metronidazole, and a ninhydrin assay for tobramycin.

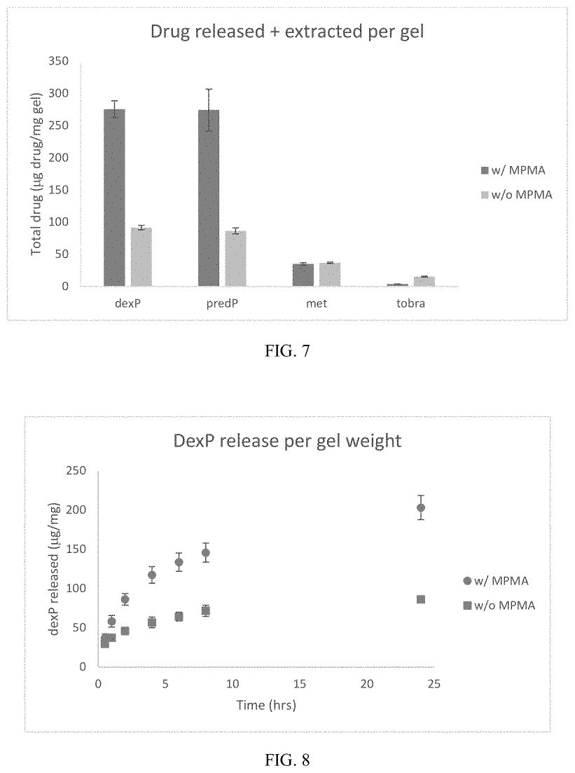

As seen in FIG. 7, the total amount of drug released and extracted was significantly greater for the negatively charged drugs, dexP and predP, when MPMA was incorporated in the crosslinked material. For the neutral-charge drug, metronidazole, the presence of MPMA in the matrix did not affect the amount released and extracted. For the positively-charged drug, tobramycin, the presence of MPMA in the matrix significantly decreased the amount of drug released and extracted. The total amount of drug released and extracted for metronidazole was lower than for dexP and predP, which may be due to the decreased concentration of drug in the soaking solution (10 mg/ml for metronidazole vs 40 mg/ml for the other drugs) due to the lower water solubility of metronidazole, thereby resulting in a decreased amount of drug loaded.

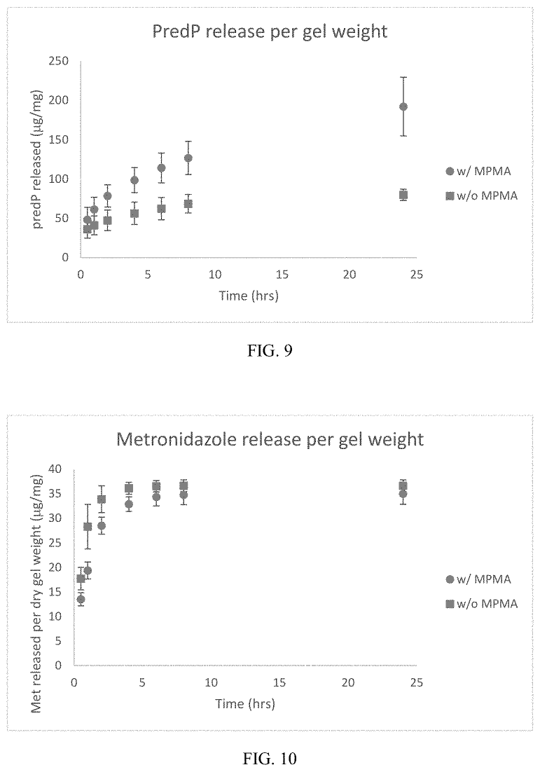

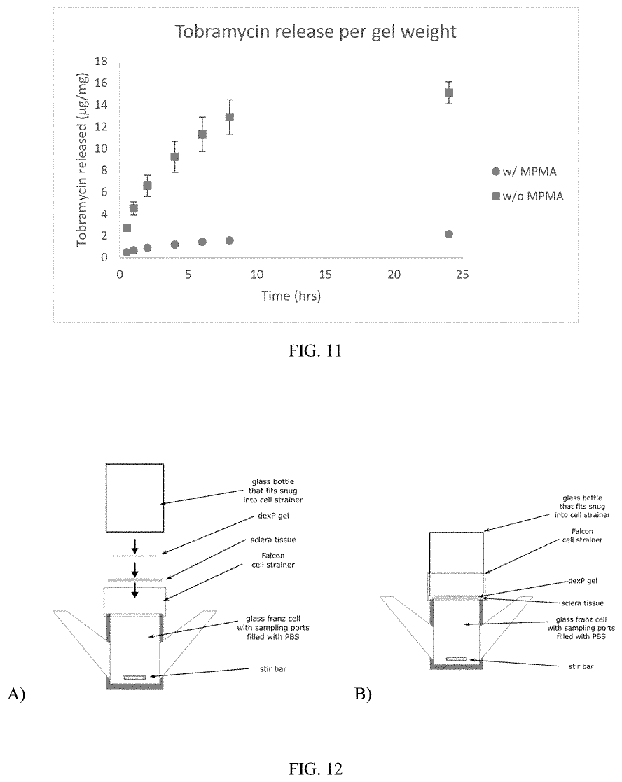

Drug release profiles over time are shown in FIGS. 8-11. As seen in FIGS. 8 and 9, the dexP and predP are released in controlled manner over at least 24 hours when MPMA is incorporated into the crosslinked material, but the release is substantially lower after just 2-4 hours when MPMA is not incorporated. The incorporation of MPMA into the crosslinked material has very little effect on the release of metronidazole, with very similar release profiles with and without MPMA, as shown in FIG. 10. Release of tobramycin is significantly reduced when MPMA is incorporated into the crosslinked material, affecting both the total amount of drug released and the release profile over time, as shown in FIG. 11.

### Example 7: In Vitro Drug Release into Sclera Tissue

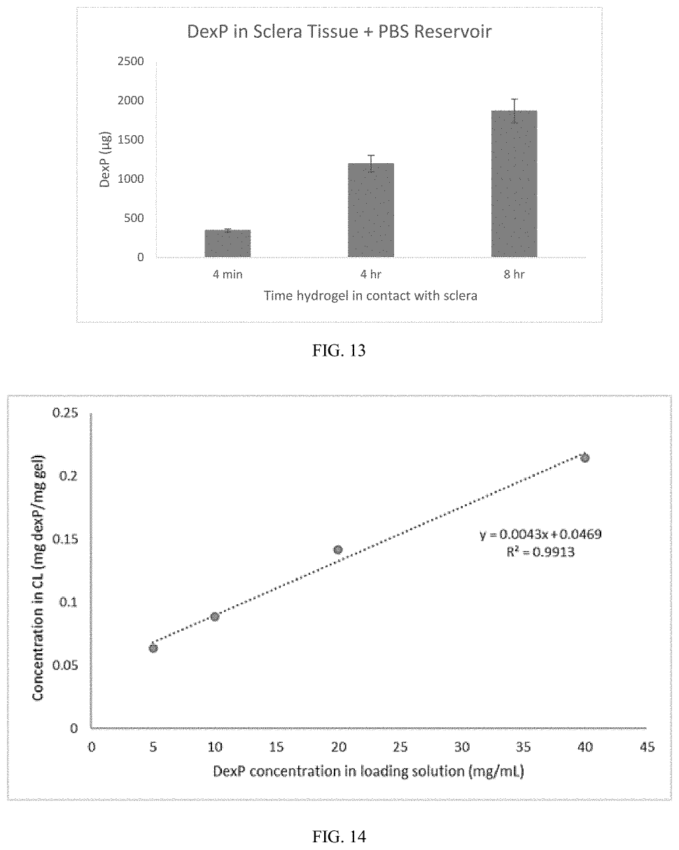

Discs loaded with dexP were made as described in Example 4, except that the discs were 17 mm in diameter and were not dried after soaking in the drug solution. An experimental setup (shown in FIG. 12) for determining drug release from a soft contact lens hydrogel disc into and across sclera tissue was created using a glass Franz cell. The Franz cell was filled with PBS. A cell strainer (BD Falcon, 70 mm nylon, 23 mm ID) was fitted onto the Franz cell as a framework to hold the sclera and hydrogel disc together. Porcine sclera tissue (21 mm diameter, 1 mm thick) was placed in the cell strainer, the drug-loaded hydrogel disc was placed onto the sclera tissue, and a glass bottle was placed on top of the disc to firmly hold the assembly together in the cell strainer. At 4 minutes, 4 hours, or 8 hours, the sclera tissue was removed and a sample of the PBS from the Franz cell was taken. The sclera tissue was digested using collagenase, and the amount of dexP in the digested sclera tissue and PBS was determined using HPLC-UV. There was an increasing amount of dexP in the sclera tissue and PBS over time, as shown in FIG. 13.

### Example 8: Effect of Loading Solution Concentration on Drug Loading and Release from Contact Lenses

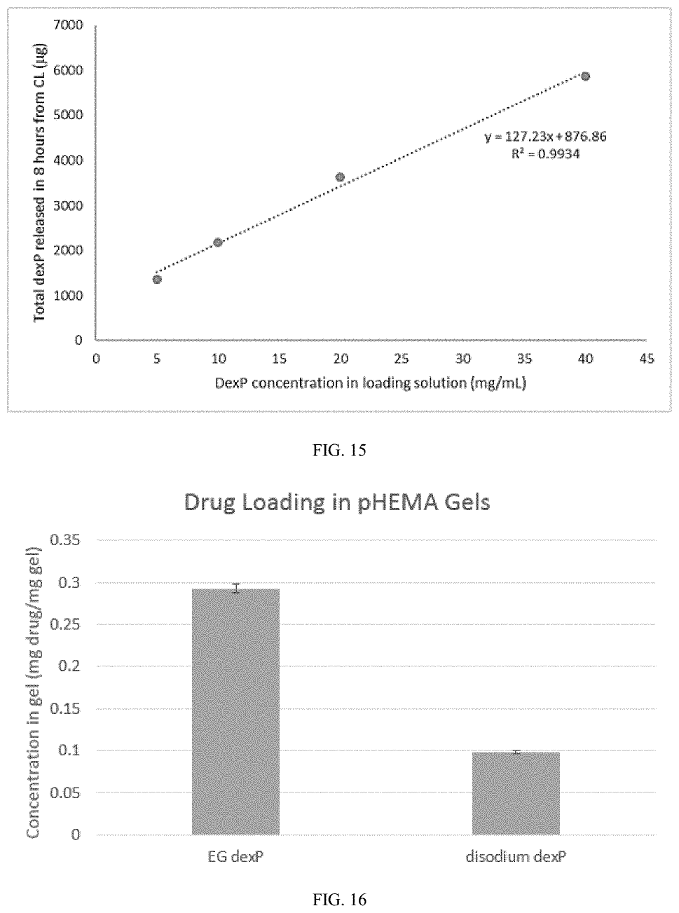

HEMA-based hydrogels were made as described in Example 4 using Formulation 3. A loading and release study was performed as described in Example 4 for dexamethasone phosphate (dexP), while the concentration of dexP in the loading solution was varied (40 mg/mL, 20 mg/mL, 10 mg/mL, and 5 mg/mL of dexP) to assess the potential for varying the amount of drug loaded and delivered. The results of the study, as shown in FIGS. 14 and 15, showed a linear relationship between the concentration of dexP in the loading solution and the amount actually loaded into the CL material as well as the amount released.

### Example 9: Loading and Release Using Different Forms of Dexamethasone Phosphate

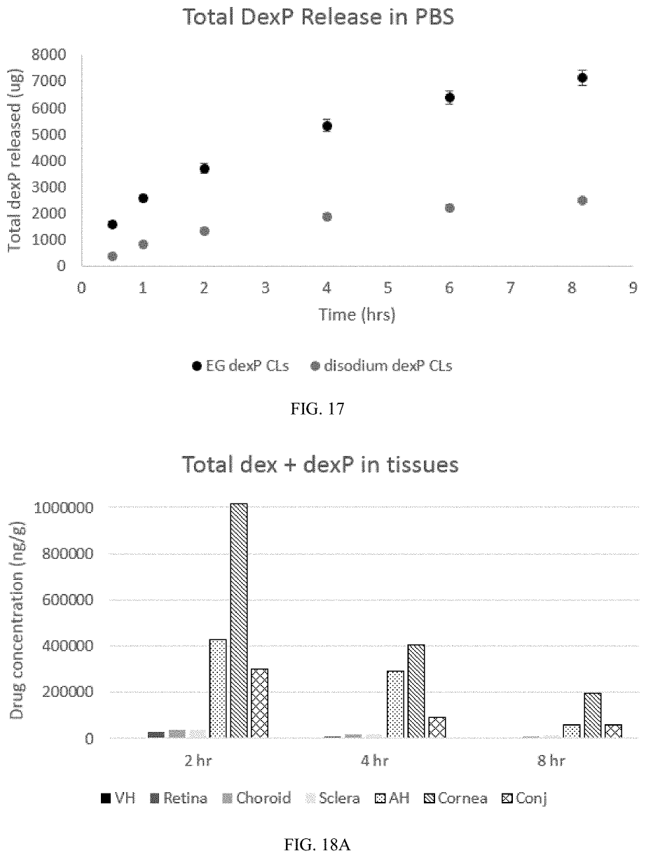

HEMA-based hydrogels were prepared as described in Example 4 using Formulation 3. A loading and release study was performed as described in Example 4 for dexamethasone phosphate (dexP), where the hydrogel discs were soaked for 7 days in a 40 mg/ml solution of either dexP free acid or disodium dexP. The discs were then placed in PBS for up to 8 hours, and the amount of dexP released over time assessed. The total amount of dexP released per weight of the disc was also determined. As shown in FIGS. 16 and 17, the additional ions present in the disodium dexP solution appeared to limit the ionic interactions with the MPMA monomer in the contact lens material, resulting in decreased loading and release of dexP with the disodium form compared to the free acid form.

### Example 10: In Vivo Drug Distribution Following Release from Contact Lenses

Formulation 3 was used to create contact lenses, placing the liquid material in UV-transparent contact lens molds and exposing to UV light (365 nm) for approximately 10 minutes to crosslink the material. Contact lenses were removed from the molds, and unreacted components were extracted as described in Example 4. Contact lenses were then placed in glass bottles with water and autoclaved at 121° C. for 18 minutes to sterilize them. Following sterilization, lenses were handled aseptically. Contact lenses were loaded with dexamethasone phosphate by soaking the lenses as described in Example 4, and were stored in sterile dexamethasone phosphate solution at 4° C. until use (dexP-loaded lenses). Control (drug-free) lenses were soaked and then stored (at 4° C.) in sterile isotonic saline until use. Controls were used only for retention and ocular health assessments.

A total of 11 New Zealand White rabbits were used in this study: 2 animals were in the control group and 9 animals in the treatment group. The nictitating membranes of the rabbits were removed and the rabbits allowed to recover for 2 weeks prior to study initiation. For the study, a contact lens was placed on each eye of an animal and a tarsorrhaphy performed by placing a single staple in the corner of the eyelids to aid in retention of the contact lenses. Cage side observations of the animals occurred every 2 hours to monitor lens retention and ocular health. At 2, 4, and 8 hours following lens placement, 3 animals in the treatment group were euthanized, the staple and contact lens from each eye were removed, and ocular health was noted. The following tissues were then isolated: aqueous humor (AH), vitreous humor (VH), retina, choroid, cornea, conjunctiva, and sclera. The aqueous humor was retrieved from each eye using a sterile 30-gauge needle on a 1 mL syringe while the eyes were still intact in the animal. Tissues were transferred to pre-weighed, pre-labeled tubes in a box of dry ice. Tissue samples were then transferred to a −80° C. freezer until further processing. Tissue samples were analyzed using LC-MS/MS for dexamethasone and dexamethasone phosphate concentration.

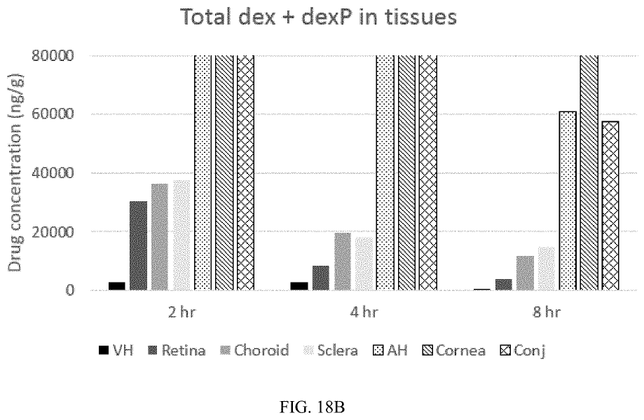

No cage side issues were observed during the study. Some discharge was noted at the 8-hr timepoint for the control and treatment animals (but not at the 2-hr or 4-hr timepoints) and is likely due to the imperfect fit of the contact lenses on the rabbit eyes. Dexamethasone and dexamethasone phosphate were detected in the tissues of treated animals at each timepoint, as shown in FIGS. 18A and 18B. Drug concentrations were highest in the front of eye tissues (AH, cornea, conjunctiva) as expected for drug being delivered onto the surface of the eye. Drug concentrations in the tissues were highest at the 2-hr timepoint and decreased over time; this is also expected based on the drug release profiles from Example 6. Despite the decrease in concentration over time, the continued release of the drug over the 8 hours on the eye allows for extended presence of the drug compared to a single large bolus of drug delivered to the surface of the eye. Additionally, the continued release allowed for continued movement of the drug through the ocular tissues to reach the back of the eye, as seen by the drug concentrations observed in the VH, retina, and choroid, and is not typically observed when delivering a bolus of drug to the surface of the eye.

1. A contact lens comprising:
a drug delivery portion which includes
a covalently crosslinked polymer;
a monomer having the formula X—(CH₂)ₙ—Z, wherein X is a crosslinkable group; n is 2, 3, or 4; Z is a morpholino, imidazole, or piperazine group; wherein the monomer is covalently linked to the crosslinked polymer through X;
and a therapeutic molecule having a negative charge;
wherein the contact lens has from about 20 wt % to about 40 wt % water.

2. The contact lens of 1, wherein X is a methacryl, an acryl, a methacrylamide, an acrylamide, or a vinyl group.

3. The contact lens of claim 1, wherein the monomer is at a concentration of about 5 wt % to about 20 wt %.

4. The contact lens of claim 1 wherein the polymer is covalently crosslinked using light.

5. The contact lens of claim 4 wherein the light has a wavelength of about 200 nm to about 400 nm.

6. The contact lens of claim 1, wherein the therapeutic molecule is a corticosteroid.

7. The contact lens of claim 1, wherein the therapeutic molecule is a dexamethasone derivative.

8. The contact lens of claim 1, wherein the therapeutic molecule has a phosphate group.

9. The contact lens of claim 1, wherein the therapeutic molecule has a concentration of about 1% to about 5%.

10. The contact lens of claim 1, wherein the contact lens has a thickness between about 0.1 mm and about 0.5 mm.

11. The contact lens of claim 1, wherein the polymer comprises HEMA.

12. The contact lens of claim 1, wherein the polymer comprises a silicone macromer.

13. The contact lens of claim 1, wherein the therapeutic molecule is incorporated into the drug delivery portion prior to crosslinking.

14. The contact lens of claim 1, wherein the therapeutic molecule is incorporated into the drug delivery portion by soaking the contact lens in a solution containing the therapeutic molecule.

15. The contact lens of claim 14, wherein the therapeutic molecule in the solution is at a concentration of about 5 to about 80 mg/ml.

16. The contact lens of claim 15, wherein the therapeutic molecule in the solution is at a concentration of about 5 to about 40 mg/ml.

17. The contact lens of claim 16, wherein the therapeutic molecule is dexamethasone phosphate.

18. The contact lens of claim 1, wherein the therapeutic molecule is incorporated into the drug delivery portion prior to crosslinking and also by soaking the contact lens in a solution containing the therapeutic molecule.

19. The contact lens of claim 18, wherein the therapeutic molecule is dexamethasone phosphate.

20. The contact lens of claim 1, wherein the drug delivery portion is a circumferential portion of the contact lens.
